(19) United States
(12) Patent Application Publication
(10) Pub. No.: US 2017/0368244 A1
(43) Pub. Date: Dec. 28, 2017

(54) DEVICES FOR INFANT FEEDING

(71) Applicants: The Trustees of Columbia University in the City of, New York, NY (US); RAMOT at Tel-Aviv University Ltd., Tel-Aviv (IL)

(72) Inventors: David Elad, TEL AVIV (IL); Andrew Francis Laine, New York, NY (US); Catherine Watson Genna, WOODHAVEN, NY (US); Uri Zaretsky, KDUMIM (IL); Pavel Kozlovsky, HOLON (IL)

(21) Appl. No.: 15/701,011

(22) Filed: Sep. 11, 2017

Related U.S. Application Data
(63) Continuation of application No. PCT/US16/21740, filed on Mar. 10, 2016.
(60) Provisional application No. 62/131,549, filed on Mar. 11, 2015.
(60) Provisional application No. 62/278,322, filed on Jan. 13, 2016.

(51) Int. Cl. A61M 1/06

(52) U.S. Cl. CPC A61M 1/066; A61M 2205/50; A61M 2205/3306; A61M 2205/3344

(57) ABSTRACT

A system for infant feeding adapted to mimic an infant's mouthing and suckling during breastfeeding.

### CROSS REFERENCE TO RELATED APPLICATIONS

This application is a continuation of International Application No. PCT/US16/21740, filed Mar. 10, 2016, which claims the benefit of U.S. Provisional Patent Application Nos. 62/131,549, filed Mar. 11, 2015 and 62/278,322 filed Jan. 13, 2016, the contents of which are incorporated herein by reference.

### FIELD

The present disclosure relates to improved devices for breastfeeding, and more particularly provides improved breast pumps, measurement devices, and artificial nipples adapted to mimic an infant's mouthing and suckling during breastfeeding.

### BACKGROUND

Breastfeeding is widely encouraged because of the benefits breast milk provides to infants. Breast milk is known to provide nutrients and immunities required for growth and development during the first months after birth. Successful breastfeeding requires the infant to latch onto the breast and nipple so that the nipple, areola, and underlying mammary tissue and lactiferous ducts are drawn into the infant's mouth with the nipple tip extended as far as the hard-soft palate junction. For latch-on, the infant attaches his lips and tongue tip to the areola and generates oral sub-atmospheric pressure to extend the nipple and part of the areola into his mouth until about the hard soft palate junction, which is about 25 mm from the lips for many subjects, although it varies based on individual physical characteristics. Additionally, active manipulation of the mandible and tongue provide compression of the areola and the underlying ducts to extract the milk into the mouth of the infant. During breastfeeding, a continuous seal is maintained between the infant lips and the breast while tongue undulation and mandible oscillations generate the mechanisms required to extract milk from the breast and to swallow.

Recent simulations suggest that appropriate latch-on requires a sub-atmospheric pressure of about −20 mmHg, while nutritive breastfeeding requires oscillating oral sub-atmospheric pressure in the range −20 mmHg to −40 mmHg.

Although breastfeeding has its advantages, it also has limitations. Lactating mothers sometimes need to be away from the infant because of employment or other commitments. Other times, mother and infant aren't in a private setting for comfortable breast feeding opportunities. During such situations, breastfeeding may become unmanageable. Some mothers' plan ahead and use breast pump systems to extract milk beforehand, and store it for later use. Although conventional breast pumps provide suction, they do not provide other physical dynamics comparable to infant breastfeeding. This results in drawbacks including suboptimal milk expression, or long periods of time of pumping to obtain sufficient milk supply, and the mother does not become used to the sensations of infant feeding on her breast.

Another drawback for breast feeding mothers is the infant's nipple confusion for those mothers that choose to breast feed and supplement by bottle feeding. The artificial nipple of a bottle is different in configuration and texture than the mothers' nipple and the infant sometimes chooses one or the other leading to unsuccessful breast feeding or bottle feeding. Conventional artificial nipples do not have the mechanical properties comparable to a mother's nipple. As a result, a baby may experience reductions in masseter strength and nipple confusion, when moving from bottle to breast, thereby compromising his ability to feed from his mother.

Also, about 5-10% of newborn infants have difficulty breastfeeding due to the anatomy/physiology of the infant's mouth and/or the mother's breast. Objective monitoring devices and efficient intervention tools are currently not available.

Accordingly, there remains a need for improved breast pump systems that accurately simulate in vivo breastfeeding for the most optimal extraction of milk, artificial nipples that inhibit nipple confusion, as well as diagnostic and monitoring tools for breastfeeding.

### SUMMARY

In one aspect of the present disclosure, a device for milk expression is provided. The device includes a conical lumen adapted to receive a nipple, such as a breast cup portion. An inflatable element is arrayed on an interior surface of the conical lumen. A vacuum source is in fluid communication with a distal end of the conical lumen. A pressure source is in fluid communication with the inflatable element. Some embodiments include a controller adapted to adjust a pressure of the pressure source at a predetermined frequency and to adjust a negative pressure of the vacuum source at a predetermined frequency. In some embodiments, the inflatable element comprises silicone. Some embodiments include a milk receptacle, such as a bottle portion, in fluid communication with the distal end of the conical lumen. Some embodiments include a concave portion extending from an exterior circumference of a proximal end of the conical lumen.

The controller may adjust positive pressure output from the pressure source at a first predetermined frequency and negative pressure from the vacuum source at a second predetermined frequency. In some embodiments, the first and second predetermined frequencies are concurrent. The controller may housed within the second device or may be remote from the second device and provided in a third stand-alone device. In some embodiment, the controller is communicatively coupled to a computer system through a data acquisition module.

In one embodiment, the pressure source may be physically or operatively connected to the inflatable element by tubing. The vacuum source may be physically or operatively connected to the conical lumen by tubing. The inflatable element cyclically inflates and deflates over a period of time. The vacuum source exerts negative pressures in the conical lumen of about −20 to −40 mmHg. The amount of negative pressure exerted in the conical lumen periodically increases and decreases over time. The amount of negative pressure exerted in the conical lumen is cyclical.

In some embodiments, the inflatable element is a silicone sleeve. The breast cup portion and the bottle portion are configured to detachably couple. The breast cup portion includes a concave portion extending from an exterior circumference of a proximal end of the conical lumen.

In another aspect of the present disclosure, a diagnostic device is provided. The diagnostic device includes a conical lumen adapted to receive a nipple. A photodetector is arrayed longitudinally along an interior surface of the conical lumen. A light emitter is arrayed longitudinally along the interior surface of the conical lumen. The light emitter and photodetector are substantially opposite each other. A vacuum source is in fluid communication with a distal end of the conical lumen. In some embodiments, the diagnostic device includes a controller adapted to adjust a negative pressure of the vacuum source. In some embodiments, the diagnostic device includes a signal processor adapted to receive a signal from the photodetector, the signal indicative of an obstruction of a line of sight between the light emitter and the photodetector. In some embodiments, the diagnostic device includes a pressure sensor coupled to the conical lumen.

In yet another aspect of the present disclosure, a device for infant feeding is provided. The device includes a nipple having a length. The nipple has elasticity such that the length approximately doubles when exposed to a pressure of −20 mmHg to −40 mmHg.

It is to be understood that both the foregoing general description and the following detailed description are exemplary and are intended to provide further explanation of the disclosed subject matter claimed.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a schematic view of a breast pump system according to various embodiments of the present disclosure in use.

FIG. 2 depicts an exemplary sinusoidal relationship between the pressure exerted in the inflatable element of a breast pump according to various embodiments of the present disclosure and bottle pressure.

FIG. 3 depicts exemplary vertical mouth width and sucking pressure of an infant during breastfeeding.

FIG. 4 depicts a breast cup portion and bottle portion according to an embodiment of the present disclosure.

FIG. 5 depicts an exploded view of the components of the FIG. 4.

FIG. 6 is a cross-sectional view of the components of FIG. 4.

FIG. 7A depicts an insert for the breast pump of FIG. 4 according to an embodiment of the present disclosure.

FIG. 7B is a cross-sectional view of the insert of FIG. 7A.

FIGS. 8A-8C depict the assembly of the insert of FIG. 7A with the breast pump of FIG. 4 according to an embodiment of the present disclosure.

FIG. 9 is a photograph of the insert of FIG. 7A.

FIG. 10A depicts the components of another embodiment of a breast cup portion and bottle in accordance with the described subject matter.

FIG. 10B depicts a cross section of the breast cup portion and bottle of FIG. 10A.

FIG.. 11a show specifications of one embodiment of an insert in accordance of the described subject matter.

FIG. 11b show specifications of another embodiment of an insert in accordance of the described subject matter.

FIG. 12 depicts an exemplary comparison between latch-on and peak suckling of a breastfeeding model and a breast pump according to the inserts shown in FIGS.. 11a and 11b.

FIG. 13A to 13C depicts schematically drawings of a mold that can be used in the manufacture of the insert of the described subject matter.

FIG. 14 depicts an exploded view of the mold of FIG. 13A.

FIG. 15 is a schematic view of another embodiment of a breast pump system according to the present disclosure.

FIG. 16 is a schematic view of a breast pump controller according to an embodiment of the present disclosure.

FIG. 17 is a schematic view of a valve controller according to an embodiment of the present disclosure.

FIG. 18 is a schematic view of another embodiment of a breast pump system controller according to the present disclosure.

FIG. 19 depicts exemplary nipple elongation during latch-on for various nipple elastic properties and oral sub-atmospheric pressures.

FIG. 20A depicts a nipple according to an embodiment of the present disclosure.

FIG. 20B is a side view of the nipple of FIG. 20A

FIG. 21A is a cross-sectional view of the nipple of FIG. 20A while undeformed.

FIG. 21B is a cross-sectional view of the nipple of FIG. 20A during latch-on.

FIG. 21C is a cross-sectional view of the nipple of FIG. 20A at maximum extension during suckling.

FIG. 22 is a schematic view of a latch-on meter controller according to an embodiment of the present disclosure.

FIG. 23 is an exploded view of a latch-on meter controller of FIG. 22 according to an embodiment of the present disclosure.

FIG. 24 is a schematic view of a combined breast pump controller and latch-on meter controller according to an embodiment of the present disclosure.

FIG. 25 depicts exemplary output of a latch-on meter according to the present disclosure

FIG. 26 is a schematic view of a latch-on meter controller according to an embodiment of the present disclosure.

FIG. 27 is a schematic view of a combined breast pump controller and latch-on meter controller according to an embodiment of the present disclosure

FIG. 28 depicts an exemplary comparison between latch-on and peak suckling of a breastfeeding model and a breast pump according to embodiments of the present disclosure.

FIG. 29 depicts exemplary tongue kinematics during bottle feeding.

FIG. 30 depicts exemplary tongue kinematics during breastfeeding.

FIG. 31 illustrates another embodiment of a latch on meter.

FIG. 32 shows an exploded view of the latch-on meter of FIG. 31.

### DETAILED DESCRIPTION OF EXEMPLARY EMBODIMENTS

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

Reference will now be made in detail to exemplary embodiments of the disclosed subject matter. Methods and corresponding steps of the disclosed subject matter will be described in conjunction with the detailed description of the system.

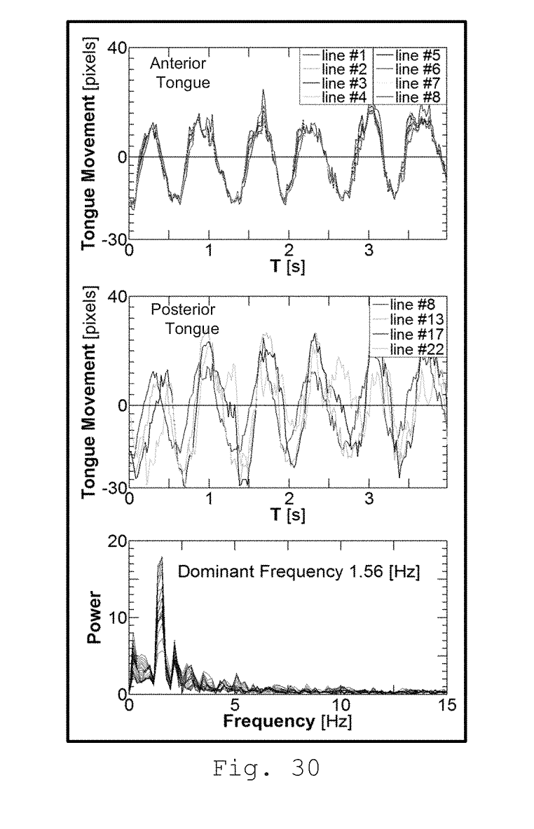

Generally, a breast pump system configured to mimic the actions of an infant during breast feeding is provided. The breast pump system provides improved pumping of breast milk because it is adapted to operate similar to the physical principles associated with an infant's oral cavity during successful, natural, breastfeeding. FIGS. 29 and 30 show the kinematics of an infant's tongue movement and power during natural breastfeeding. In this regard, the breast pump system is configured to simulates the oral sub-atmospheric pressures required for latch-on by a newborn infant, and sub-atmospheric pressure oscillations or cycles needed to successfully extract milk from the breast. Accordingly, the breast pump system of the present disclosure mimics infant breastfeeding and therefore extracts more milk in a shorter period of time compared to conventional breast pumps. The breast pump also entrain the milk ejection reflex to physiologic infant suck in mothers of pre-term or neurologically impaired infants during early stages when the facial muscles of the infant have not yet gained full strength to efficiently extract milk from the breast during breastfeeding.

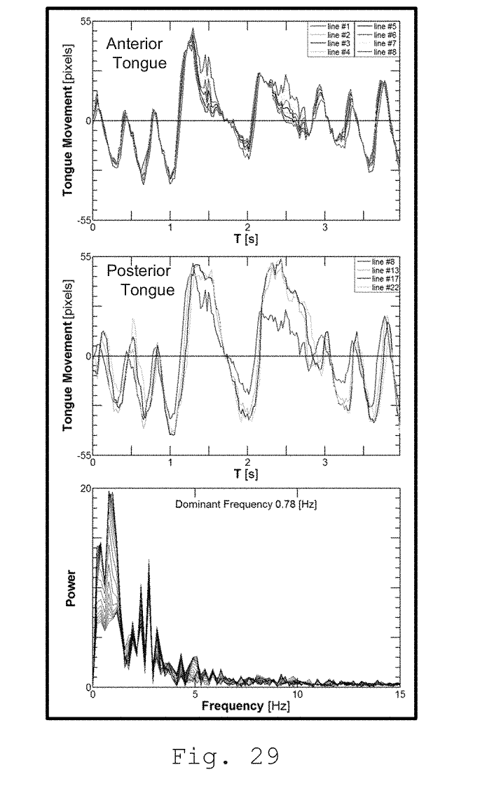

Generally, the breast pump system comprises a man-made infant mouth simulator coupled to a breast flange (breast cup portion) configured to receive the breast and a control box operatively connected to the man-made mouth simulator. The control box generally contains a vacuum source such as a vacuum pump, an air pump, valves and electronics that during operation actuate the mouth simulator to induce nipple “mouthing” and “sucking” like an infant. The mouthing and sucking mechanisms mimics in vivo breastfeeding performance. Accordingly, the breast pump system embodied herein is designed to mimic the infant's gentle nipple mouthing and sucking during in vivo breastfeeding. A comparison of in vivo breastfeeding and using the breast pump system of described and embodied herein is shown in FIG. 28. As shown and described in detail below, use of the present breast pump system mimics the mouthing and sucking of an infant resulting in teat elongation and extraction of milk.

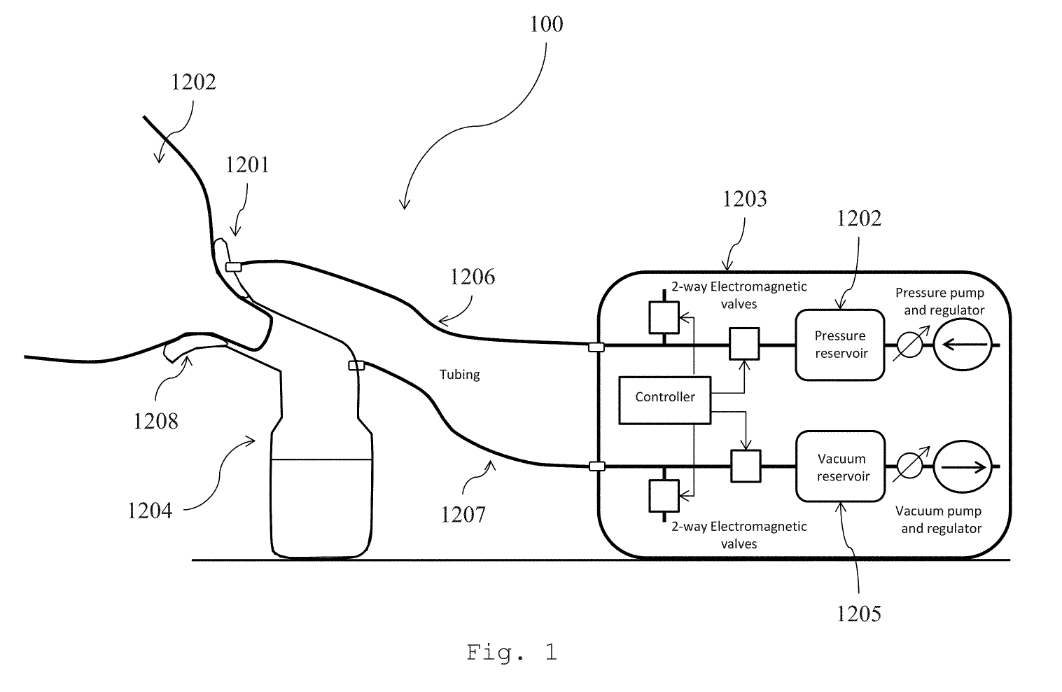

In one aspect, referring to FIG. 1, a breast pump system 100 in accordance with the disclosed subject matter comprises a man-made mouth simulator comprising a breast cup portion 1201 and bottle 1204, operatively engaged or connected to control box 1203. The breast cup portion 1201 is shaped to receive and fit against a human breast 1202 and accept nipple (and areola) into a conical lumen, as descried below in more detail. Breast cup portion 1201 includes an inflatable sleeve 1208 or element that cups the nipple and areola. The inflatable element is operatively coupled to a pressure source 1202 disposed in control box. In one embodiment, the pressure source is connected to the inflatable element by tubing 1206. A two-way valve or a check valve is opened and closed to allow pumping of air through the tubing to inflation the inflatable element. The tubing 1207 can include a dual lumen to create an air circuit so the air can travel to the inflatable member to inflate followed by deflation.

The conical lumen of breast cup portion 1201 is in fluid communication with a milk receptacle 1204 via outlets described in more detail below. The receptacle 1204 is operatively coupled to a vacuum source 1205 for example by tubing 1207. Vacuum sources can be housed within control box 1203. In operation, the inflatable sleeve or element is cyclically inflated and deflated to simulate an infant's mouthing actions during natural breastfeeding. Additionally, or concomitantly, sub-atmospheric pressure is applied to the breast via the vacuum source to extract milk from the breast and into the receptacle 1204.

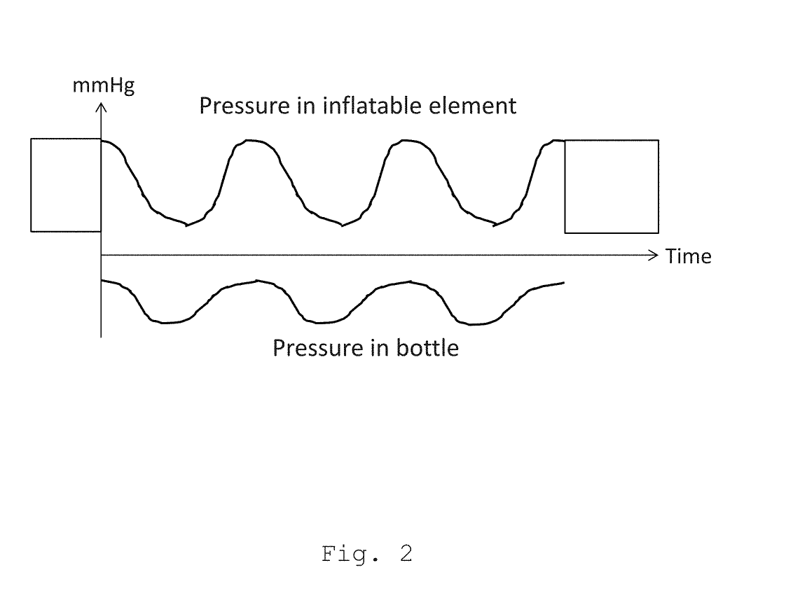

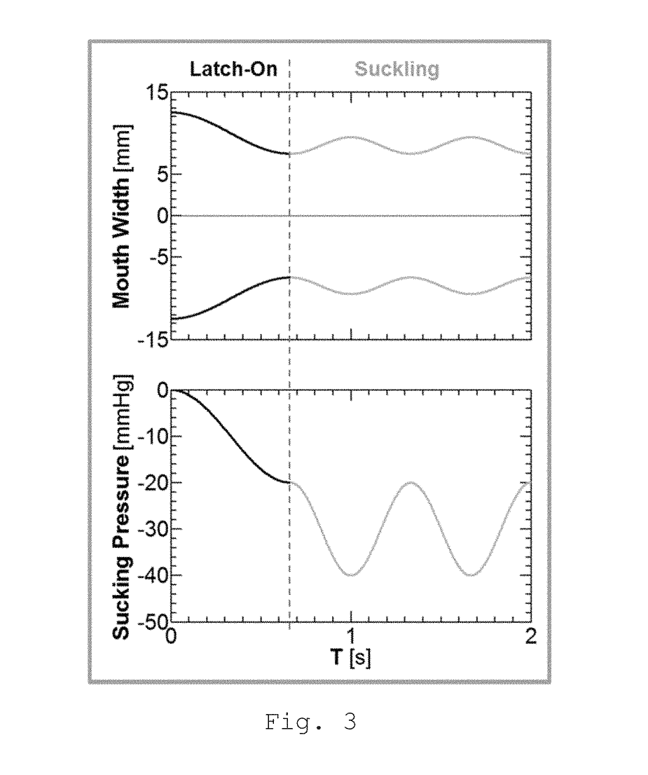

Exemplary patterns of inflation and suction in accordance with the breast pump system 100 are depicted in FIGS. 2 and 3. Referring to FIG. 2, successive or cyclical inflation and deflation of the inflatable sleeve or element 1208 of the breast cup portion 1201 is provided by a pressure source, such as a pump regulated in the control box by controller and one or more valves. The pressure source provides cycles of pressure provided according to a sinusoidal wave pattern as shown in FIG. 2. As illustrated, the pressure (mmHg) periodically increases and decreases to inflate and deflate the inflatable element. Concurrently, the pressure in the bottle follows the same pattern over time. The cyclic inflation-deflation pattern comprises uniform fluctuations of pressure that create a pattern resembling successive geometric sine waves, as depicted in FIG. 2.

The vacuum source provides cycles of negative pressure in a sinusoidal pattern so that the man-made mouth simulator, or breast cup portion, mimics the suckling action of an infant during breastfeeding. The cyclic negative pressure pattern comprises fluctuations of negative pressure that creates geometric sine waves, as shown in FIG. 3. In one embodiment, the negative pressure cycles from about −20 to −40 mmHg. Accordingly, the breast pump system is configured to mimic the nipple mouthing action that plays a role in stimulation of the lactation and let-down refluxes.

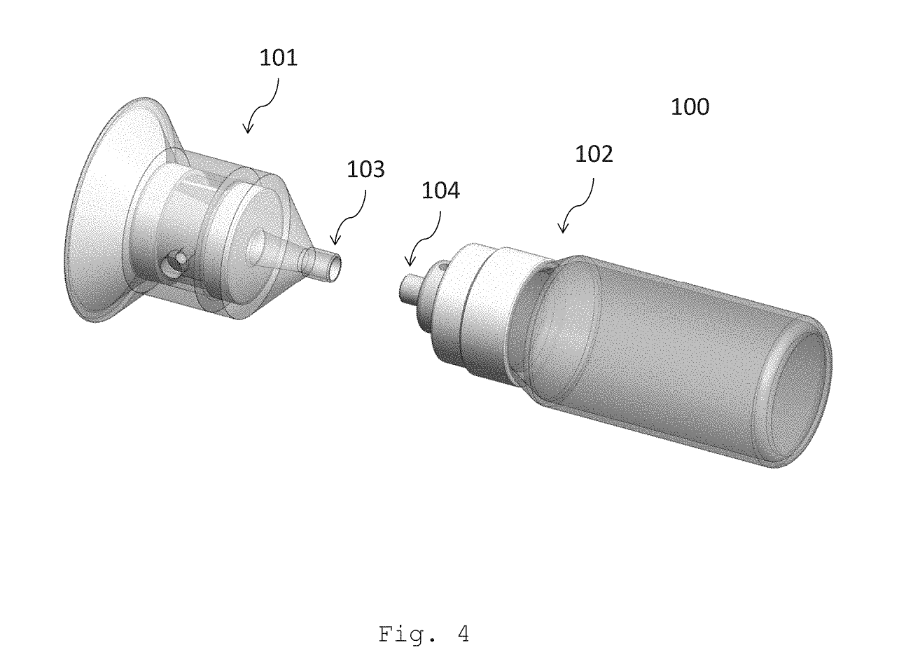

The man-made mouth simulator comprising the breast cup portion and the bottle will now be described in more detail. Referring now to FIG. 4, breast cup portion 101 and bottle portion 102 is schematically depicted. Breast cup portion 101 comprises an outlet 103 at one end and a shaped surface curving inward, e.g., concave, to receive a breast. In some embodiments, the shaped surface for cupping the breast includes inflatable sleeve or element described above. The outlet 103 of breast cup portion 101 is shaped and configured to couple with inlet 104 of receptacle, or bottle portion 102 thereby forming a physical engagement between the breast cup portion and the bottle portion.

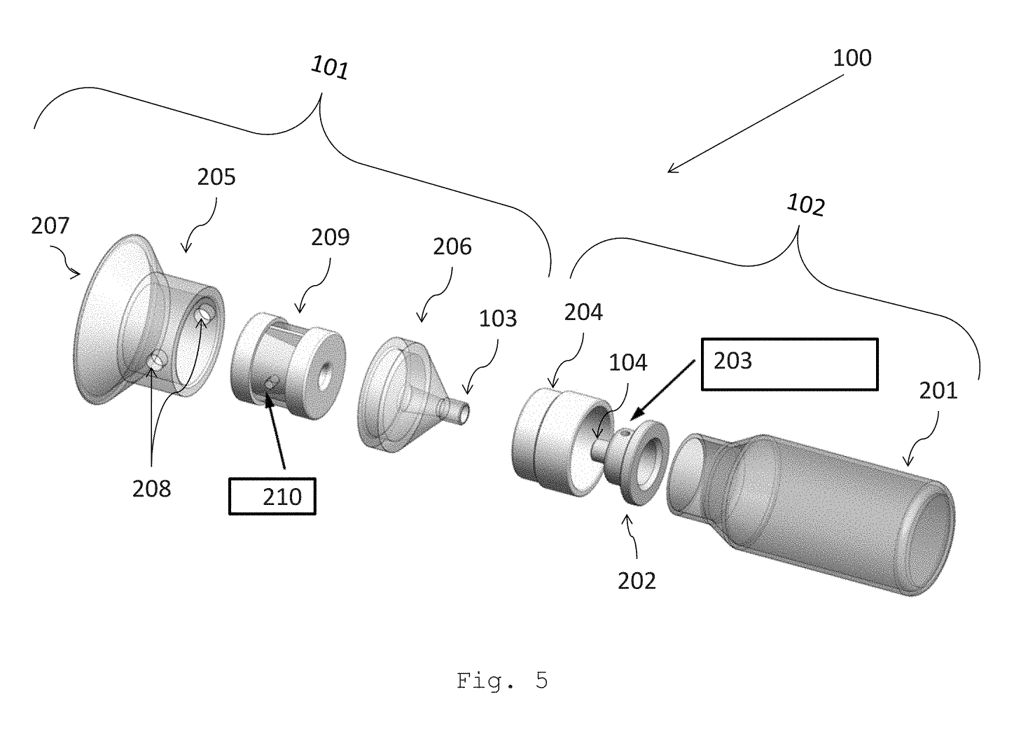

Referring to FIG. 5, an exploded view of breast cup portion 101 and bottle portion 102 are illustrated. Bottle portion 102 comprises a milk receptacle 201, cap portion 202 and retention ring 204. In one embodiment, milk receptacle 201 is a container having top and bottom ends defining a cylindrical body. The top portion of the container can have a taper or graduated surface that terminates in a neck having a smaller outer diameter than the outer diameter of the container body. The milk receptacle 201 can be made from glass, plastic, metal, or other material known in the art and is adapted to receive and store liquid. In some embodiments, the receptacle 201 is disposable.

Cap portion 202 is disposed between receptacle 201 and retention ring 204. Cap portion 202 is adapted to fit over an opening defined at the top of milk receptacle 201. Cap portion 202 includes at least one milk inlet 104 to receive milk from breast cup portion 101. Accordingly, breast cup portion 101 and a bottle portion 102. Cap portion 202 further includes vacuum inlet 203. Cap portion 202 can be made of plastic, rubber, or other material known in the art and is adapted to form an airtight seal with receptacle 201 when held in place by retention ring 204. Retention ring 204 is adapted to fit about receptacle 201 and hold cap portion 202 in place. In some embodiments, retention ring 204 is threaded and mates with corresponding threads on receptacle 201. In other embodiments, retention ring 204 fits tightly about receptacle 201 and remains in place by friction.

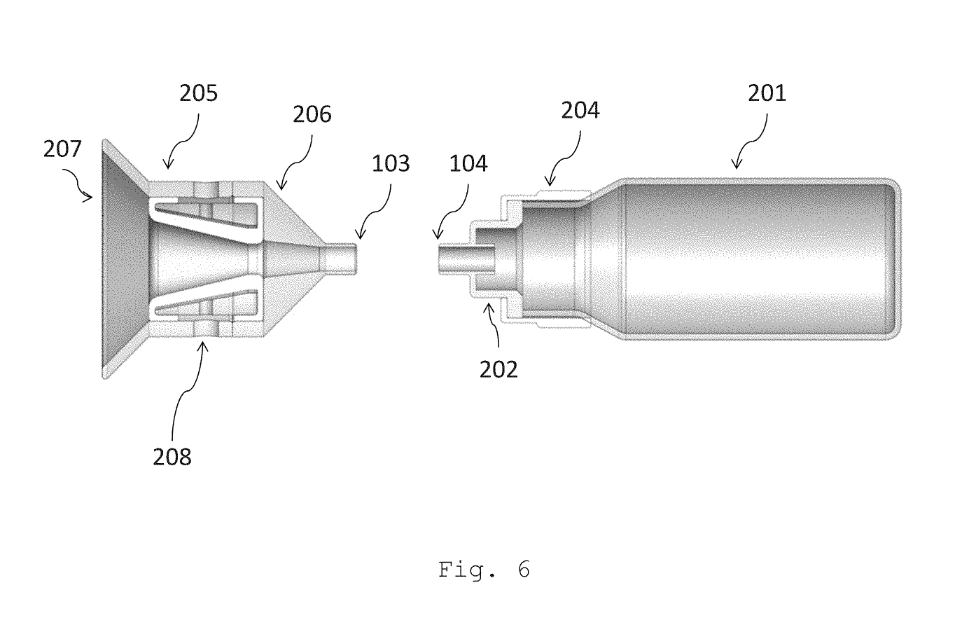

Referring back to FIG. 5, breast cup portion 101 comprises a proximal cap section 205 and a distal cap section 206. Proximal cap section 205 includes a concave portion 207 adapted to receive and fit against a human breast and encircle the nipple and areola. In one embodiment, the concave portion surface extends and engages a collar circumferentially surrounding the cap section. The collar body includes one or more ports or holes 208. Distal cap section 206 includes milk outlet 103. Distal cap section 206 has a body surface that can be conically shaped having a lower end outer diameter greater than the upper end outer diameter, and a surface wall gradually forming a taper. Proximal cap 205 is adapted to mate or physically engage to distal cap 206. In one embodiment, cartridge 209 is disposed and contained between proximal cap 205 an distal cap 206. In some embodiments, proximal cap 205 and distal cap 206 are permanently joined for example by gluing or melting. In other embodiments, proximal cap 205 and distal cap 206 are separable or removable from each other, and are joined by threads or a friction coupling. Cartridge 209 has a body a length defined by opposing ends and a lumen therebetween. Cartridge body includes one or more ports 210, which spaced on the outer surface of the cartridge body such that the one or more ports 210 align with the one or more ports 208 disposed through the surface of proximal cap 205. Accordingly, one or more inlets of cartridge 209 correspond to one or more inlets proximal cap 205. Referring to FIG. 6, a side view of breast cup portion and bottle portion is shown.

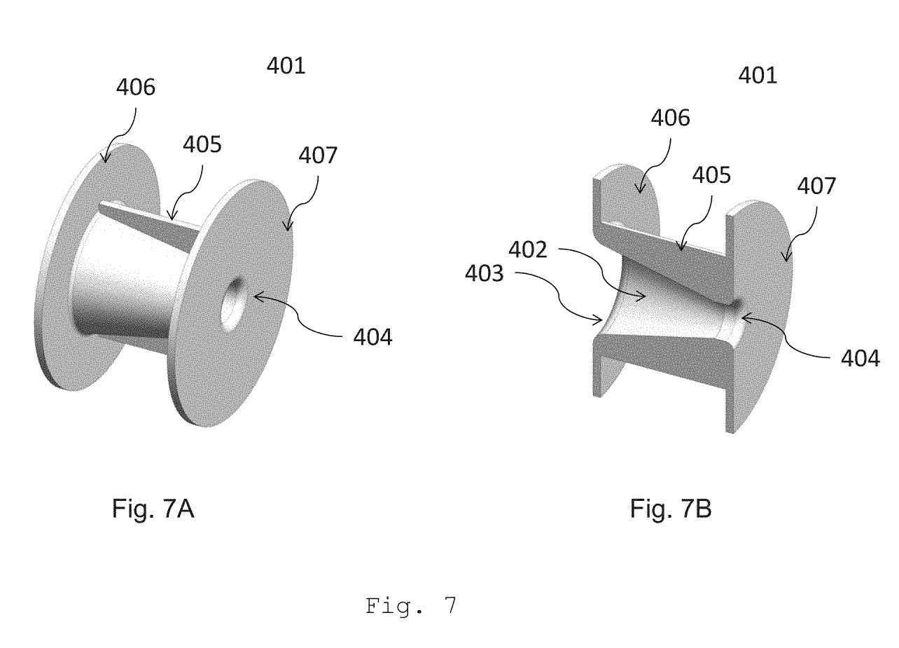

Cartridge 209 generally comprises an insert and flange. Referring to FIGS. 7A-7B, insert 401 is depicted. FIG. 7A shows a side view of insert 401, while FIG. 7B shows a cross section of the insert 401. Insert 401 can be formed from medical ultra-soft silicone (for example, MED-4086, NuSil Technology, Calif., USA). In some embodiments, insert is a silicone membrane. Insert 401 can have a substantially cylindrical body having first and second ends. The substantially cylindrical body, as shown in FIG. 7B, includes a conically shaped lumen 402 that terminates with inlet 403 and outlet 404 at opposing ends. Conical lumen 402 is sized to accept a human nipple through inlet 403. The opposing ends of substantially cylindrical body may include proximal ring 406 to distal ring 407. Proximal and distal rings, extend outwardly from the outer surface of conical body to define first and second flanges. Insert 401, in some embodiments, include one or more fins 405 disposed about and extending outwardly from the outer surface of insert body 401. The one or more fins have a longitudinal body extending from proximal ring 406 to distal ring 407.

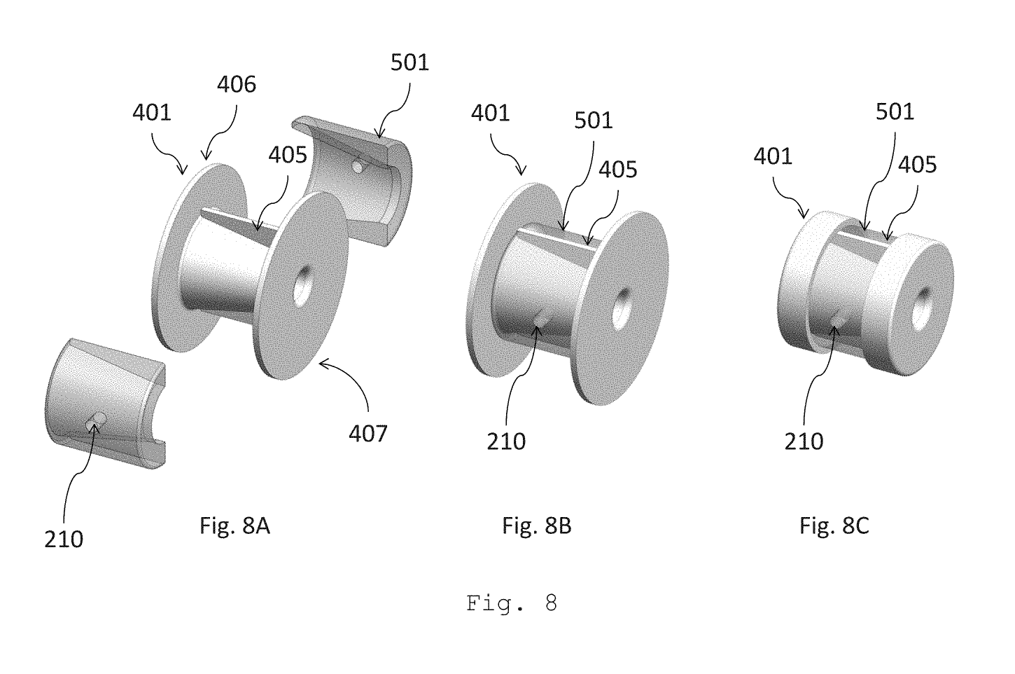

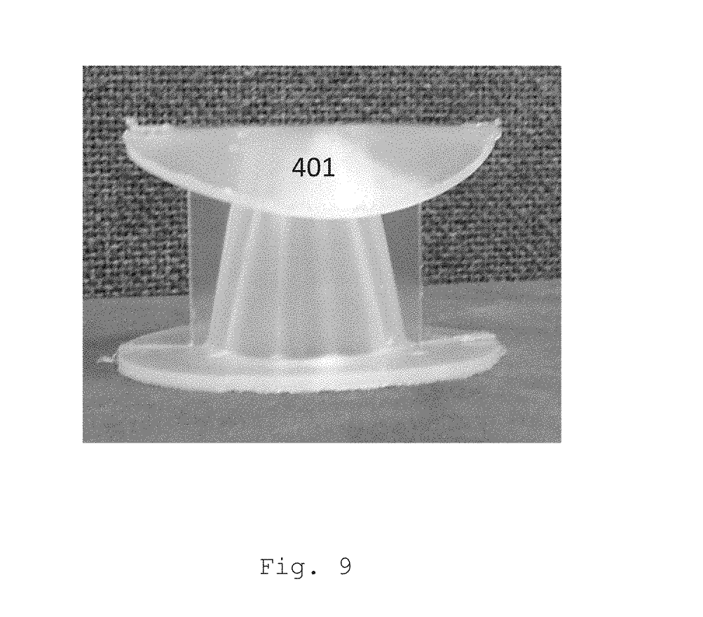

Referring to FIG. 8A-8C, flange 501 is shaped with a curved wall to encircle or surround and fit about insert body 401. In one embodiment, the flange 501 comprises multiple sections and extends between fins 405. In some embodiments, flange 501 is made of transparent plastic.

The interior surface of flange 501 has approximately the same contour or a complementary contour as the exterior surface of insert 401 and so that it may receive insert body 401 and is adapted to fit flush with insert 401 when inset 401 is not deformed. FIG. 8C depicts cartridge having engaged insert 401 and flange 501. Flange sits between proximal ring 406 and distal ring 407 with proximal and distal rings abutting flange opposing ends 501, thereby retaining flange 501 to form cartridge 209. In some embodiments, proximal ring 406 and distal ring 407 form a tight seal with flange 501 such as by, for example, heat shrinking applications. In other embodiments, a seal is formed by the pressure of caps 205 and 206. Referring to FIG. 9, an exemplary insert 401 made of soft silicone is depicted.

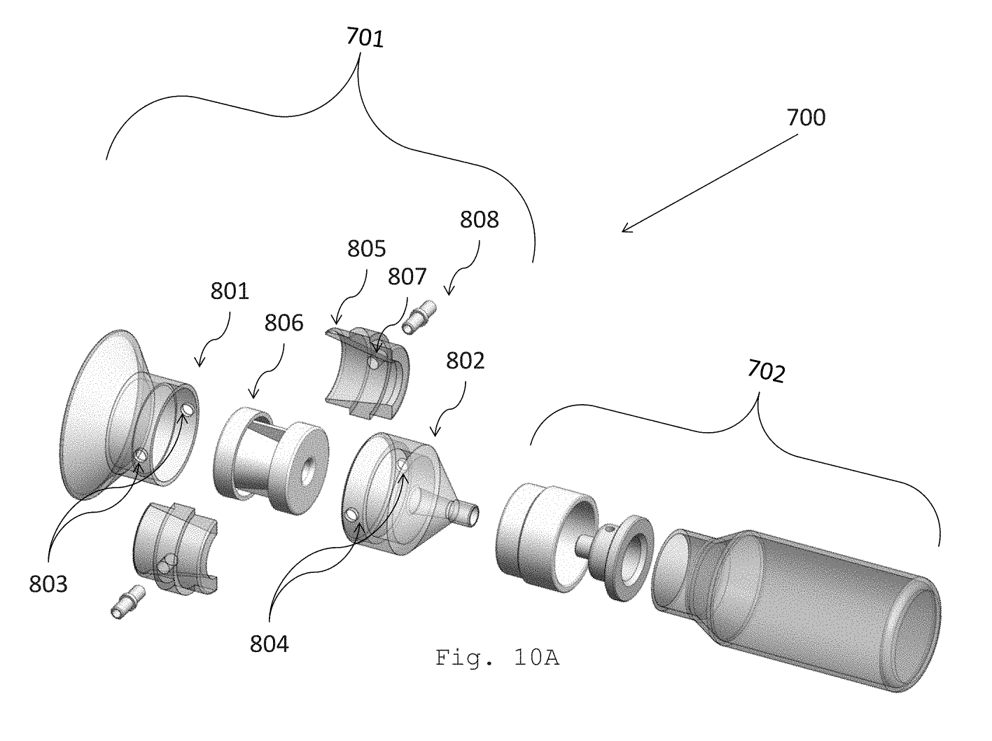

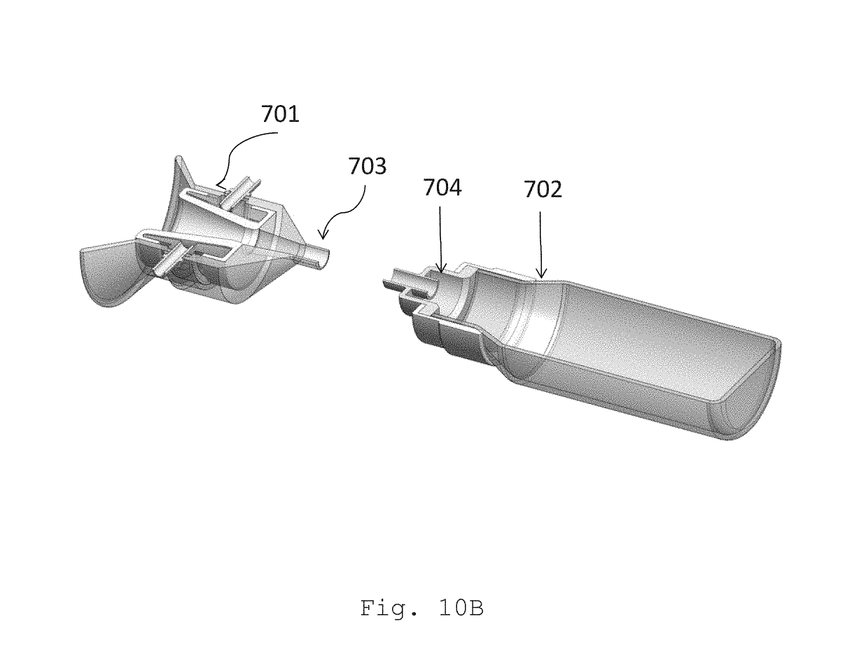

In another embodiment, as shown in FIG. 10A, breast cup portion 701 comprises a proximal cap 801 and a distal cap 802. Proximal cap 801 includes one or more ports 803. Distal cap 802 includes corresponding one or more ports 804. Distal cap 802 is adapted to extend over proximal cap 801 so that ports 803 and 804 align. Flange 805 mates with insert 806 as described above. Flange 805 includes ports 807, which align with ports 803 and 804. In this embodiment, tubular members or plugs 808 extend through ports 804, 803, and 807, holding caps 801 and 802 in place over flange 805. FIG. 10B shows a cross-sectional side view of the device of FIG. 10A.

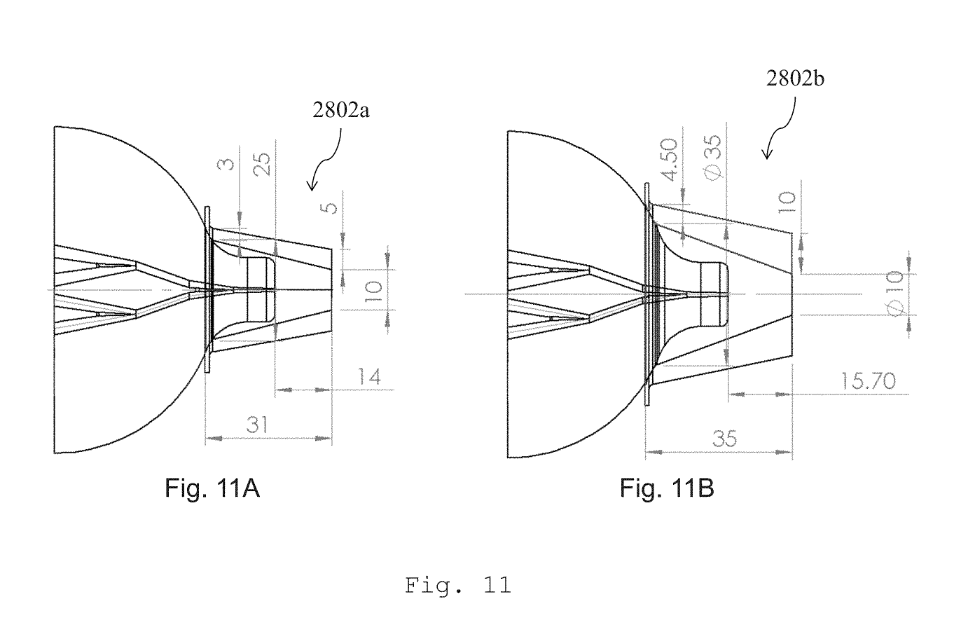

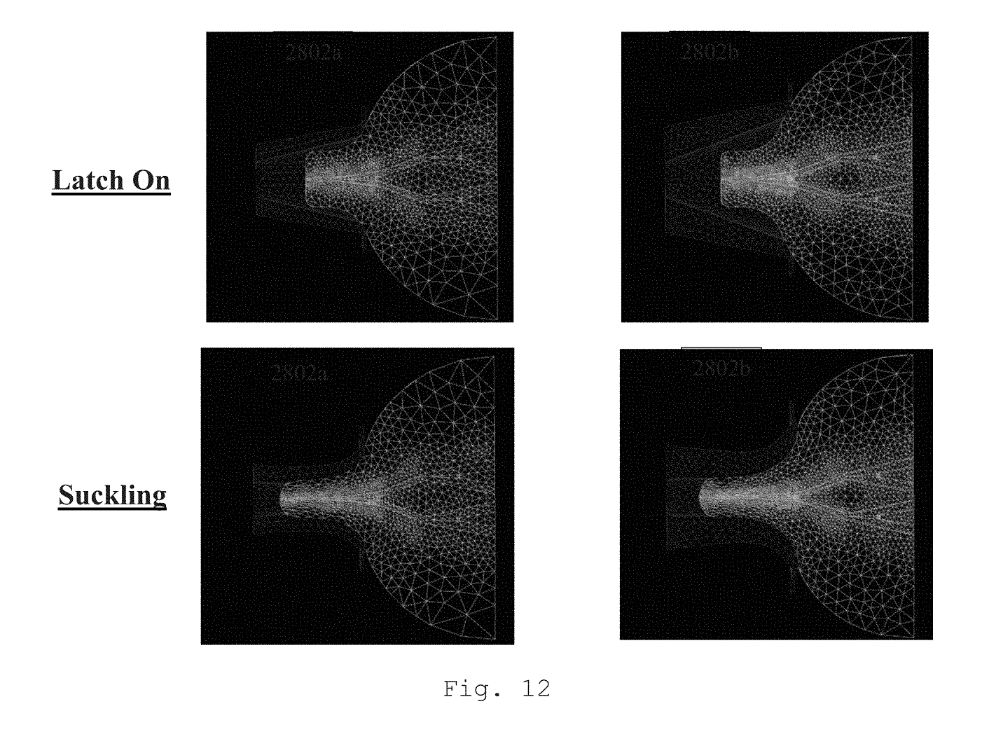

Referring to FIGS. 11a and 11b, another embodiment of insert is depicted. FIG. 11a shows a first insert 2802a, and FIG. 11b shows a second insert 2802b. Inserts 2802a and 2802b can be similar to the inserts described above (e.g., inserts 401 and 806). As shown in FIGS. 11a and 29b, the inserts 2802a and 2802b are generally similar in shape, but differ in dimension (dimensions are shown in millimeters). For example, as shown in FIGS. 11a and 11b, insert can be configured to have a length of 31 to 35 mmm and outlet with outer diameter of about 10 mm at the distal end of insert body. As shown the conical lumen of insert has a tapered wall having a thickness. The thickness at proximal end of the insert is about 3 to 4.5 mm in thickness. The spaced defined by conical lumen has a width that accommodates nipple and is about 25 to about 35 mm in length. As shown, the thickness of insert wall increases distally ending with a thickness of about 5 mm to about 10 mm. Insert is configured to have a length longer than nipple in its deformed state. In some embodiments, the nipple is received in the insert and the length of insert is about 14 to 16 mm longer than nipple length. Thus, an appropriate insert can be selected and used based on the specific characteristics of an individual's breast. Although example dimensions are shown in FIGS. 11a and 11b, these are merely illustrative examples. In practice, the dimensions and/or shapes of an insert can vary, depending on the implementation.

In some cases, inserts can differ in shape and/or size to provide an optimal latch-on and efficient milk extraction from the breast. As an example, FIG. 12 shows a comparison of latch-on and peak suckling simulation for a breastfeeding model and a breast pump using the first insert 2802a (left) and the second insert 2802b (right). As shown in FIG. 12, the inserts 2802a and 2802b each provide different mechanical characteristics during operation. Thus, an appropriate insert can be selected and used based on the specific characteristics of an individual's breast to optimize latch-on and milk extraction. As above, although example inserts and performance simulations are shown, these are merely illustrative examples. In practice, the interaction between an insert and a breast can vary, depending on the implementation.

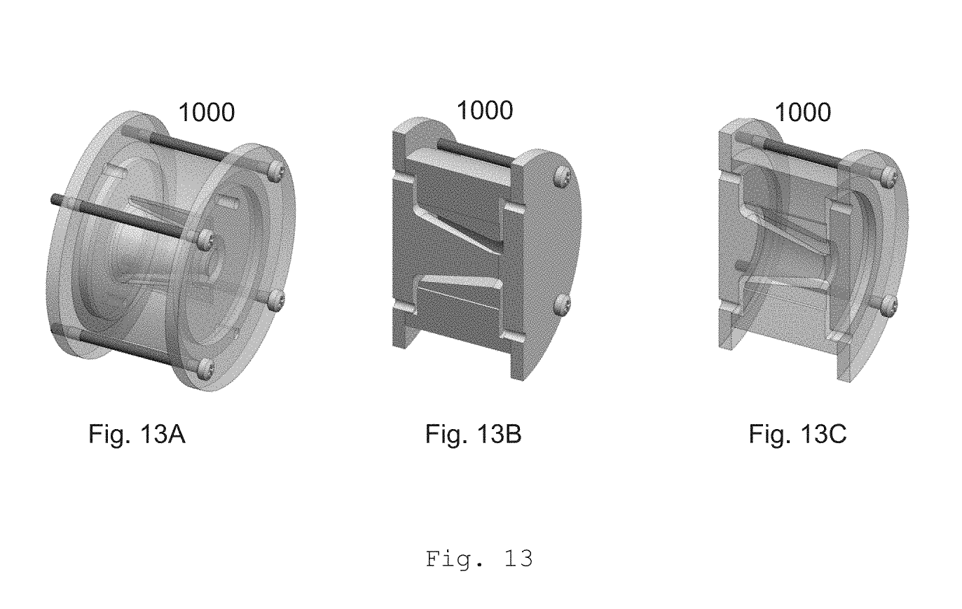

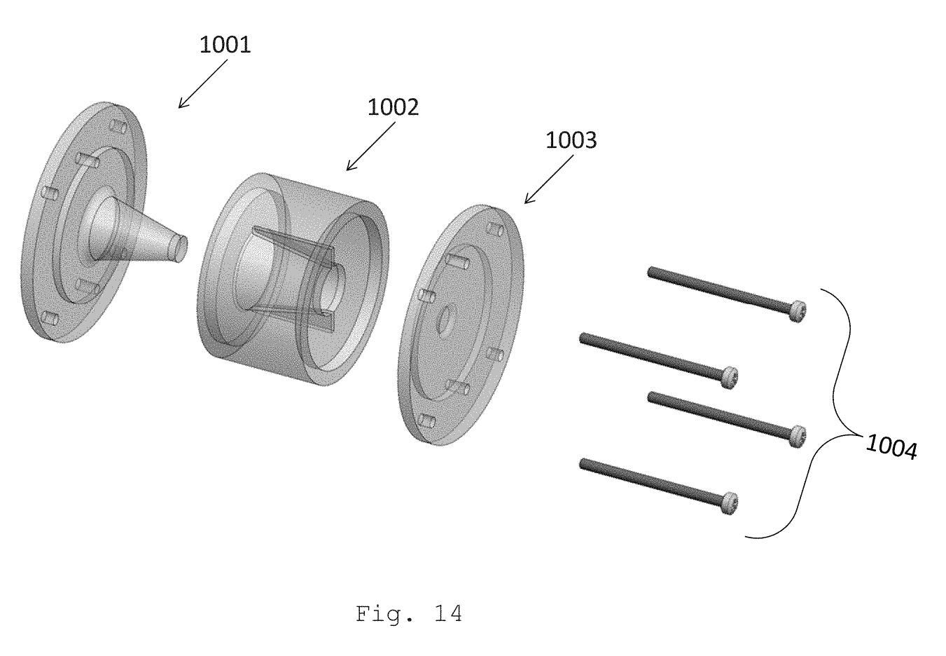

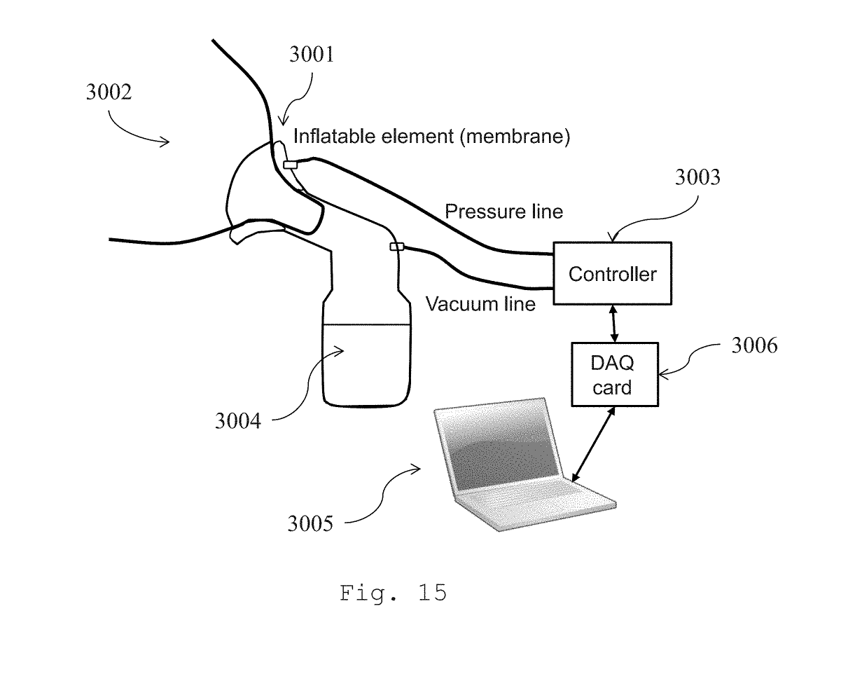

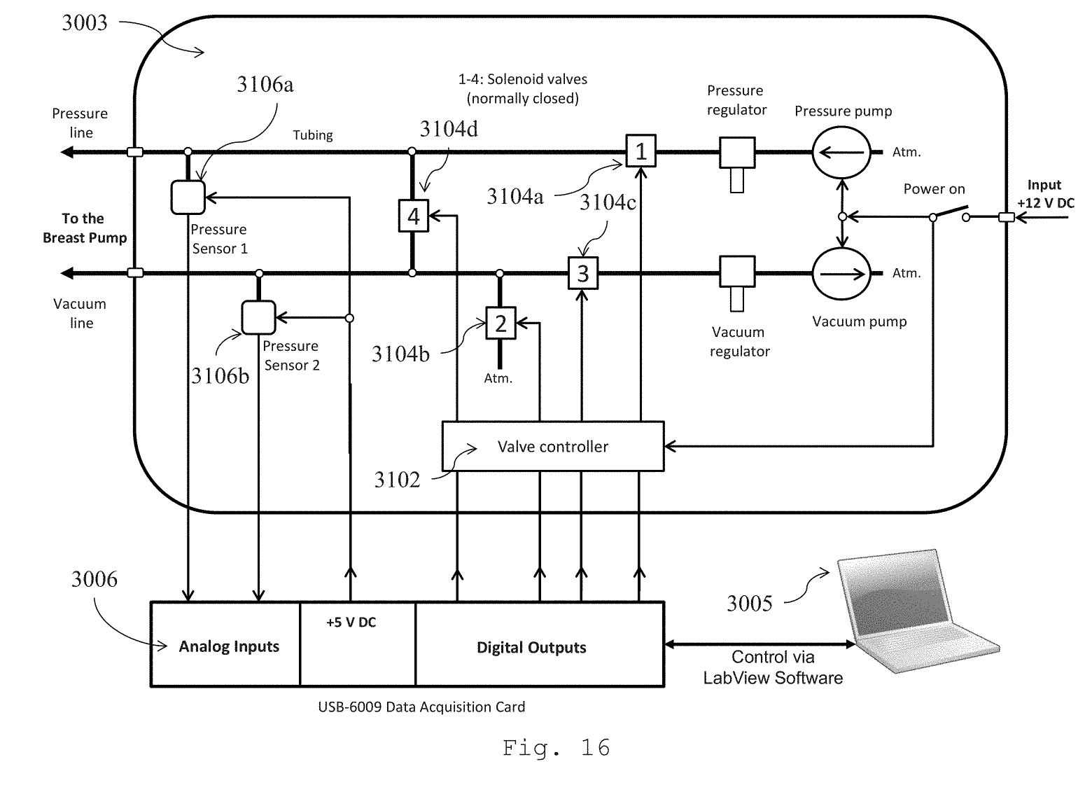

In another aspect, a method for manufacturing insert is provided. For example, the insert can be made by injection molding techniques or other molding techniques. Referring now to FIGS. 13A to 13C, various views of mold 1000 is shown. FIG. 14 depicts an exploded view of the mold shown in FIGS. 13A to 13C. As best seen in FIG. 14, the mold comprises multiple components 1001, 1002, 1003 and 1004.

In one embodiment, the method of manufacturing inserts 401 and 806 using the mold of FIG. 14 is provided. Insert can be formed from various materials using the mold 1000. As an example, the inserts can be formed from a silicone elastomer, such as MED-4086 (NuSil Technology, Calif., USA). The silicone elastomer can be prepared by mixing equal parts of MED-4086 Part A and MED-4086 Part B for a period of time (e.g., about 10 to 15 minutes), then placing the mixture in a vacuum chamber for a period of time (e.g., about 30 minutes at a vacuum of up to −300 mmHg) to remove air bubbles from the mixture. The mixture is then placed into a syringe and injected into the mold 1000 until it fill the mold 100 and appears out of the mold holes. The mold holes are then closed with plugs. The mold is placed into an oven and the mixture cured (e.g., at approximately 150° C. for approximately 45 minutes). The mold 1000 is then cooled down to room temperature, and then disassembled to extract the formed insert 401 or 806. The mold parts can be subsequently washed (e.g., with soap and hot water) and reused to form additional inserts 401 or 806. Although an example process is described above, this is merely an illustrative example. Other processes can also be used to form the inserts 401 and 806, depending on the implementation. Further, although an example material is described above, this is also merely an illustrative example. Similarly, the inserts 401 and 806 can be formed using other materials, depending on the implementation. In some cases, the inserts 401 and 806 can also include a surface treatment. As an example, the inserts 401 and 806 be coated with a silicone coating, such as MED10-6670 (NuSil Technology, Calif., USA).

The embodiments of breast pump system components can be operative connected to control box 1204 described above with respect to FIG. 1. In some embodiments, control box can be a stand-alone device that controls the breast pump system without input from any other device. The control box can control the breast pump system according to inputs received from another device, such as a computer system. In some cases, the controller can also output information to the other device. As an example, FIG. 15 shows a breast pump system. The breast pump system can be generally similar to that shown in FIG. 1 (or with embodiments described in FIGS. 4 to 10) having a cup portion 3001 that fits against a human breast 3002 and accepts a nipple into a conical lumen having an inflatable element such as the flexible inserts described above. The inflatable element is coupled to a pressure source through a controller 3003. The conical lumen is in fluid communication with a milk receptacle 3004. The receptacle 3004 is coupled to a source of vacuum through controller 3003. When in use, the inflatable element is inflated and deflated to provide a simulation of mouthing while vacuum is applied to extract milk into the receptacle 3104. Here, the controller 3003 is communicatively coupled to a computer system 3005 through a data acquisition (DAQ) module 3006. The DAQ module 3106 can be, for example, an analog/digital-digital/analog device (A/D-D/A card) coupled to the computer system 3005 via a suitable communication interface (e.g., USB, serial, Firewire, Lightning, Wi-Fi, or Bluetooth).

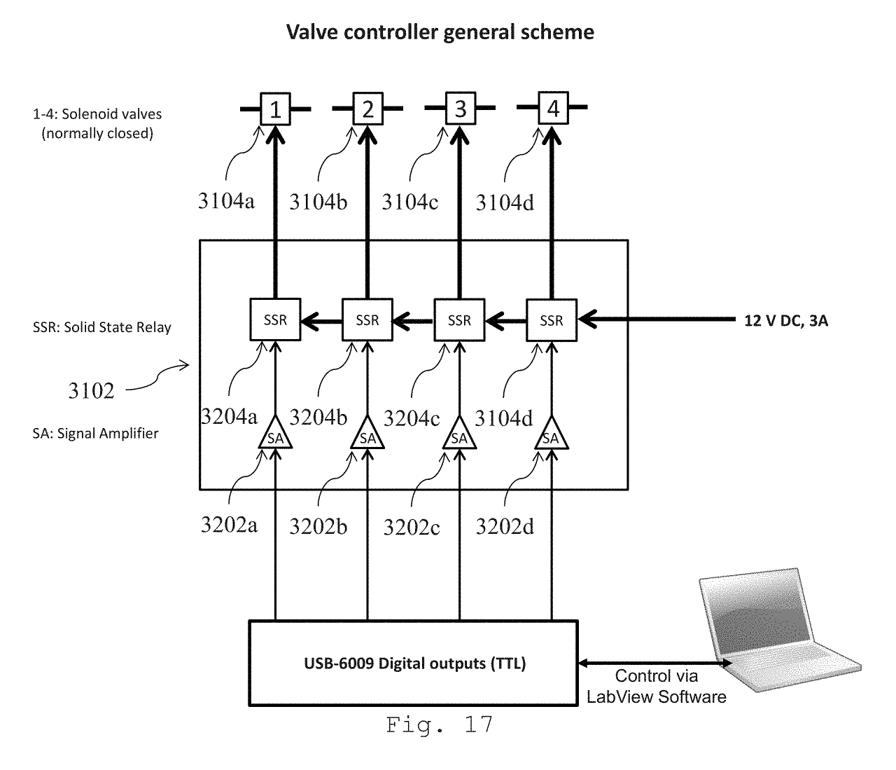

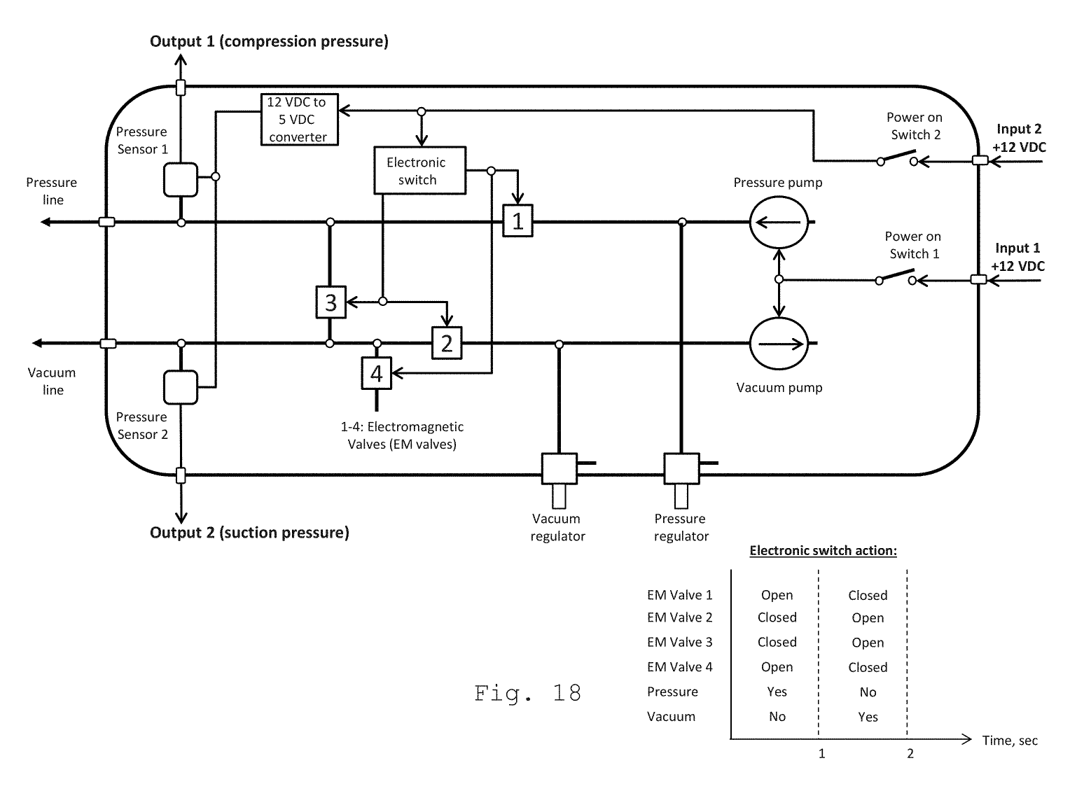

As shown in FIG. 16, in one embodiment the computer system 3005 is coupled to the DAQ module 3006, such that it can transmit commands to the controller 3003. As example, the computer system 3005 can adjust the pumping operation of the breast pump system by transmitting commands to a valve controller 3102 to control one or more valves 3104a-d. For instance, the computer system 3005 can open, close, or otherwise adjust each of the valves 3104a-d to control the pressure and/or vacuum applied by the breast pump system. As shown in FIG. 17, the valve controller can include for each of the valves 3104a-d, a signal amplifier 3202a-d and a solid state relay 3204a-d to control each respective valve 3104a-d.

Further, the computer system 3005 can receive information from one or more sensors of the breast pump system via the DAQ module 3006. As example, referring to FIGS. 16 and 17, the computer system 3005 can receive information from a sensor 3106a regarding the pressure along a pressure line and a sensor 3106b regarding the pressure along a vacuum line of the breast pumps system.

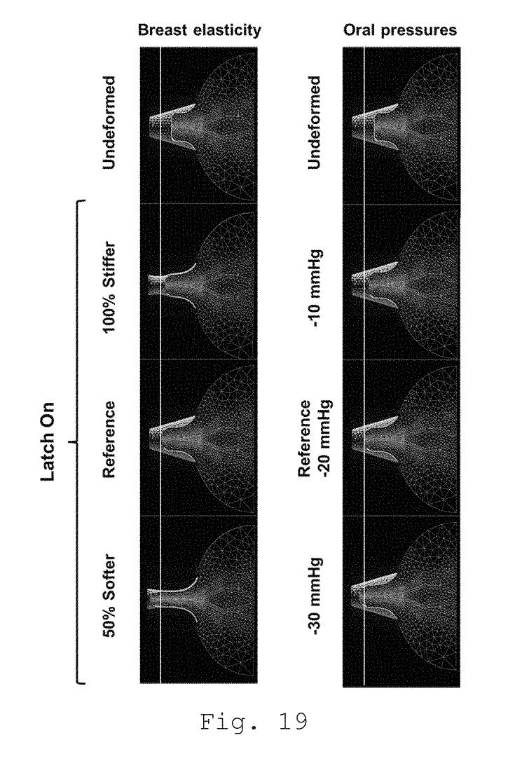

FIG. 18 depicts an exemplary breast pump controller. This controller works on 12V DC and contains a vacuum pump, an air pump, valves and an electronic circuit that operate and control the mouthing and sucking mechanisms in a way that mimics in vivo performance as set forth in further detail above.

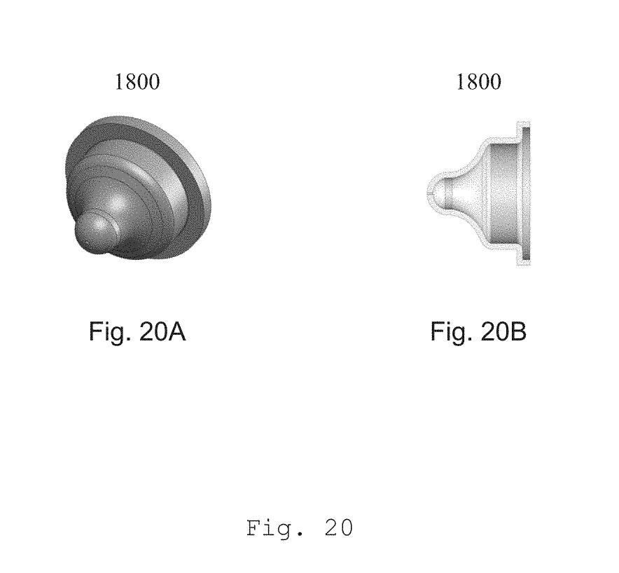

The first phase of breastfeeding requires successful latch-on during which the infant generates full contact between his tongue, lips and the mother's breast. This contact seals off the infant's oral cavity from the external environment and transforms the mother's nipple/areola into a long teat within the infant's oral cavity. During this transformation, the teat is generally about 2 times longer (or greater) than the lactating nipple at rest. Effective breastfeeding requires this transformation, i.e, formation of the teat, which is caused by the mechanical characteristics of both the mother's nipple/areola complex and the muscle power of the infant's facial muscles. Computational simulations demonstrate the expected differences in teat formation for different nipple elastic properties and oral sub-atmospheric pressures are represented in FIG. 19. FIG. 19 shows the nipple at rest in an undeformed configuration, and the differences in teat elongation when materials having different materials and durometers are used. The softer material exhibits considerably more elongation than does the reference and the stiff material. Also shown is the effect of negative pressure simulating sucking has on the elongation of the nipple. A major contributor to the nipple/areola mechanical characteristics is the skin and underlining connective structures that vary between subjects of different ethnic and racial groups. Successful in vivo breastfeeding requires latch-on that expands the nipple/areola complex (i.e., the teat) within the infant mouth to twice the length of the relaxed lactating nipple. Accordingly, in another aspect, an improved synthetic nipple is provided. Referring to FIGS. 20A and 20B, a nipple 1800 is configured to mimic the physical performance of a mother's nipple during breastfeeding. Accordingly, nipple 1800 has a geometry that mimics a typical breast, and is constructed of a soft medical polymer that allows for expansion during latch-on comparable to the natural expansion of the nipple/areola complex during in vivo breastfeeding. Nipple 1800 allows the infant to feed on a bottle in the same way that he feeds on the breast. Nipple 1800 is useful for infants with weak facial muscle or other pathologies that prohibit breastfeeding.

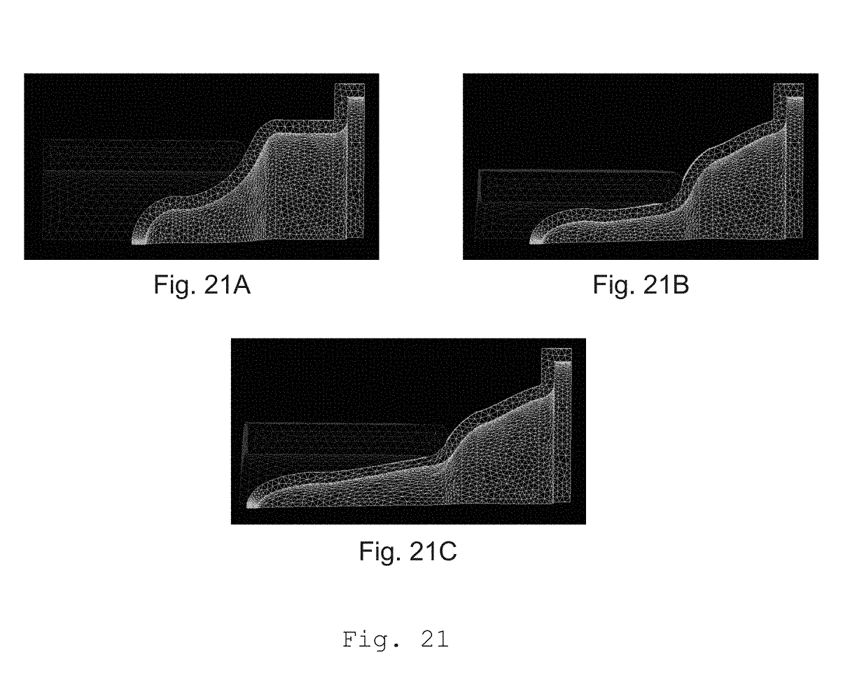

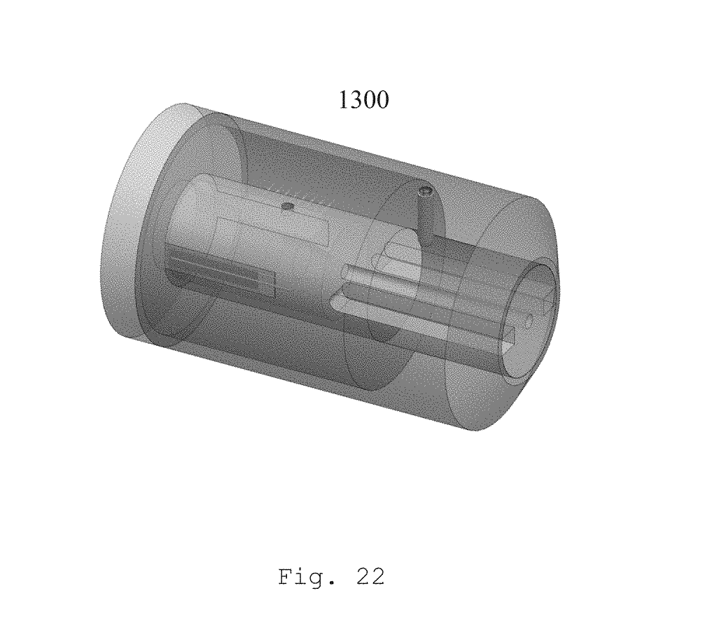

Referring to FIGS. 21 A to 21C, the extension of nipple 1800 under vacuum is illustrated. In FIG. 21A, the nipple is shown under atmospheric pressure. In FIG. 21B, the nipple is shown at −20 mmHg, which corresponds to latch-on pressure. In FIG. 21C, the nipple is shown at −40 mmHg, which corresponds to maximum suckling pressure.

In some embodiments, the nipple is formed of a silicon elastomer having a Young's modulus of about 20 kPa and a Poisson's ratio of about 0.4. For the purpose of illustration and not limitation, the nipple can be formed of the MED-4086 Ultra-Soft Low Consistency Silicone Elastomer by NuSil Technology. However, various other suitable materials are known to those of skill in the art. Conventional commercial nipples are formed from material that is too stiff and cannot be extended to the length observed in vivo by infant suckling.

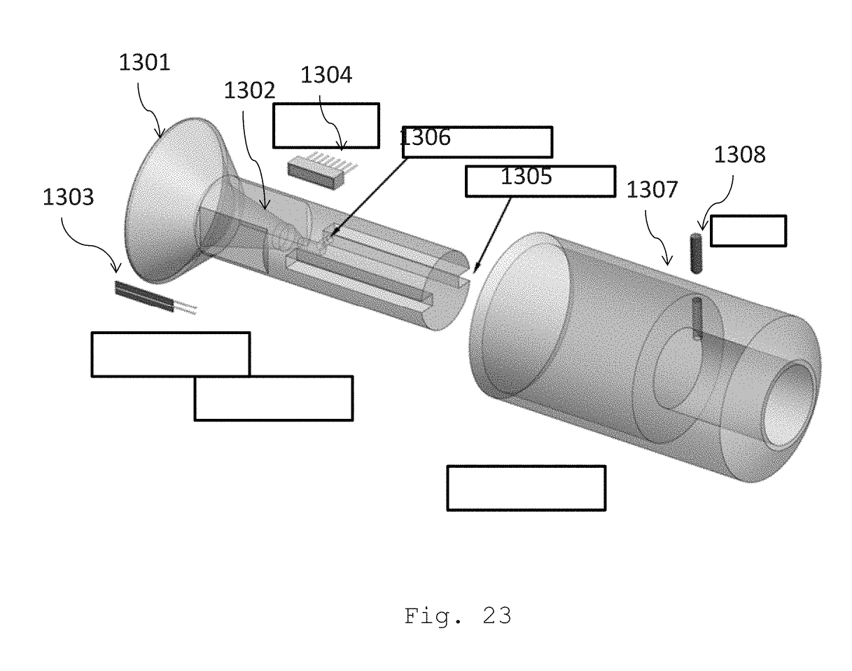

In yet another aspect, a device for non-invasive diagnosis of mechanical performance of the nipple/breast complex is provided in FIGS. 22 and 23. Device 1300 is adapted to receive a human nipple and measure its elongation under vacuum. As shown in FIG. 22 and best shown in exploded view of FIG. 23, device 1300 comprises a concave cup portion 1301 adapted to rest on the breast around the nipple. The interior of the cup portion includes a conical lumen 1302 adapted to accept the nipple/areola when sub-atmospheric is applied. A thin long photodiode 1303 is arrayed longitudinally along the conical lumen 1302. In some embodiments, the sensing surface is arranged substantially flush with the inner surface of the conical lumen. Opposite photodiode 1303, an LED array 1304 is arrayed longitudinally along the conical lumen 1302. In some embodiments, LED array 1304 includes a spherical lens adapted to generate a light surface towards the sensing surface of the photodiode. As the nipple is extended into the conical lumen 1302, it blocks part of the illumination of the plane of light from reaching the photodiode 1303. The photodiode 1303 is adapted to provide an electrical signal for direct (on-line) detection of the teat length. Progressive increase of the sub-atmospheric pressure allows for continuous acquisition of pressure versus teat length within a few minutes.

In some embodiments, wiring grooves 1305 are provided for leads to reach photodiode 1303 and LED array 1304. In some embodiments, vacuum outlet 1306 extends radially from conical lumen 1302 through the exterior surface of device 1300. In other embodiments, a vacuum outlet extends longitudinally from conical lumen 1302. In some embodiments, a shield 1307 is included around the other components and is affixed by a screw 1308.

In other embodiments, photodiode 1303 and LED array 1304 are replaced by any suitable pair of emitter and detector. For example, suitable light emitters include LEDs, other optoelectronic devices such as laser diodes, cascade lasers, or OLEDs, conventional incandescent or fluorescent bulbs, and any other light sources known in the art. In the case of a light emitter, the light source may be remote from device 1300, and directed into lumen 1302 by a lens or optical fiber. Suitable light detectors include photodiodes, CCDs, photoresistors, photovoltaic cells, phototubes, phototransitors, and various other detectors known in the art. Suitable emitter and detector pairs include those that emit and detect energy other than light, for example sounds. For example, an ultrasound emitter and detector such as those known in the art are suitable.

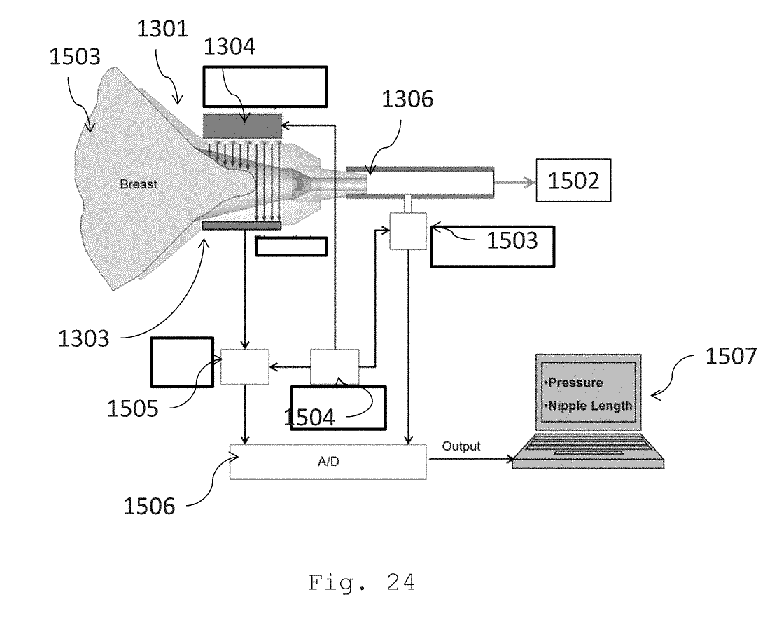

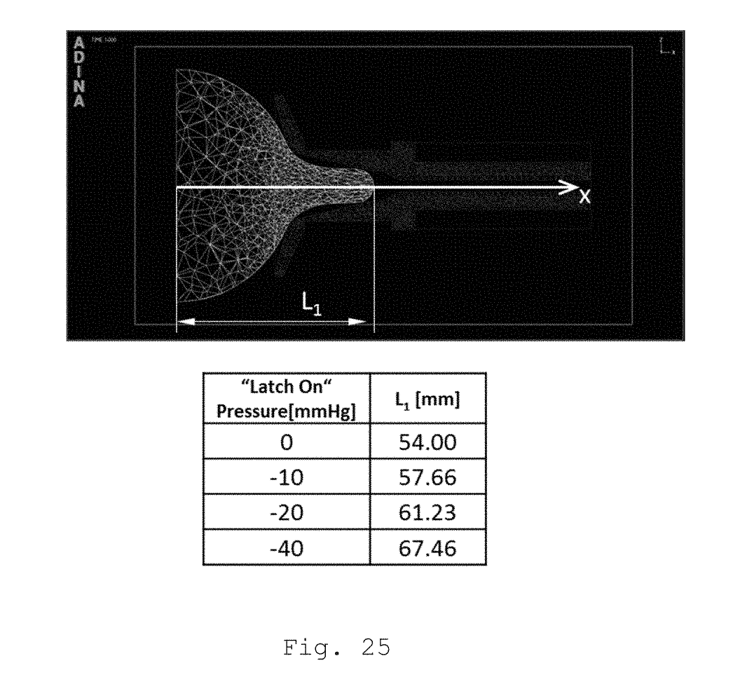

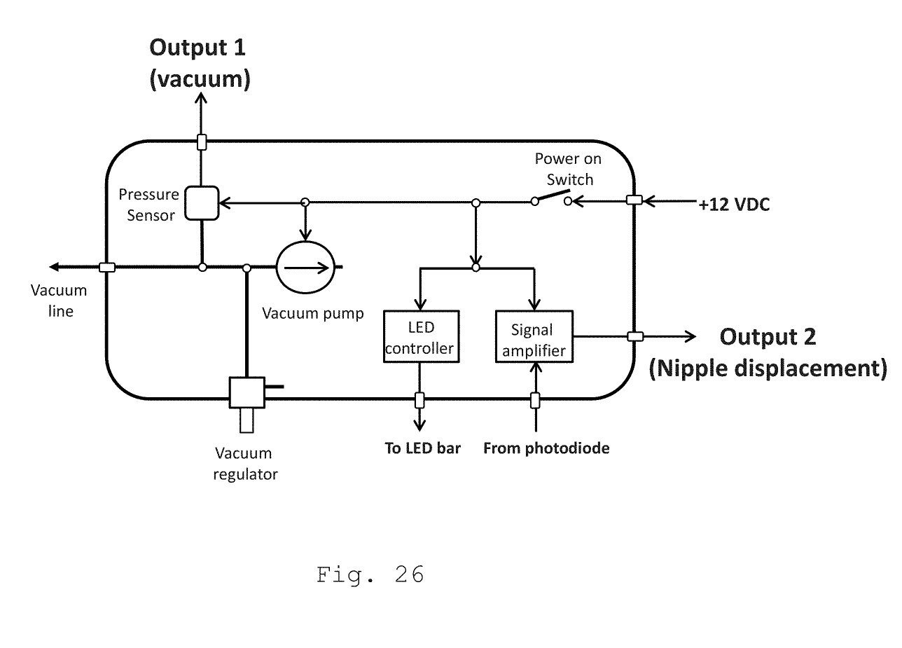

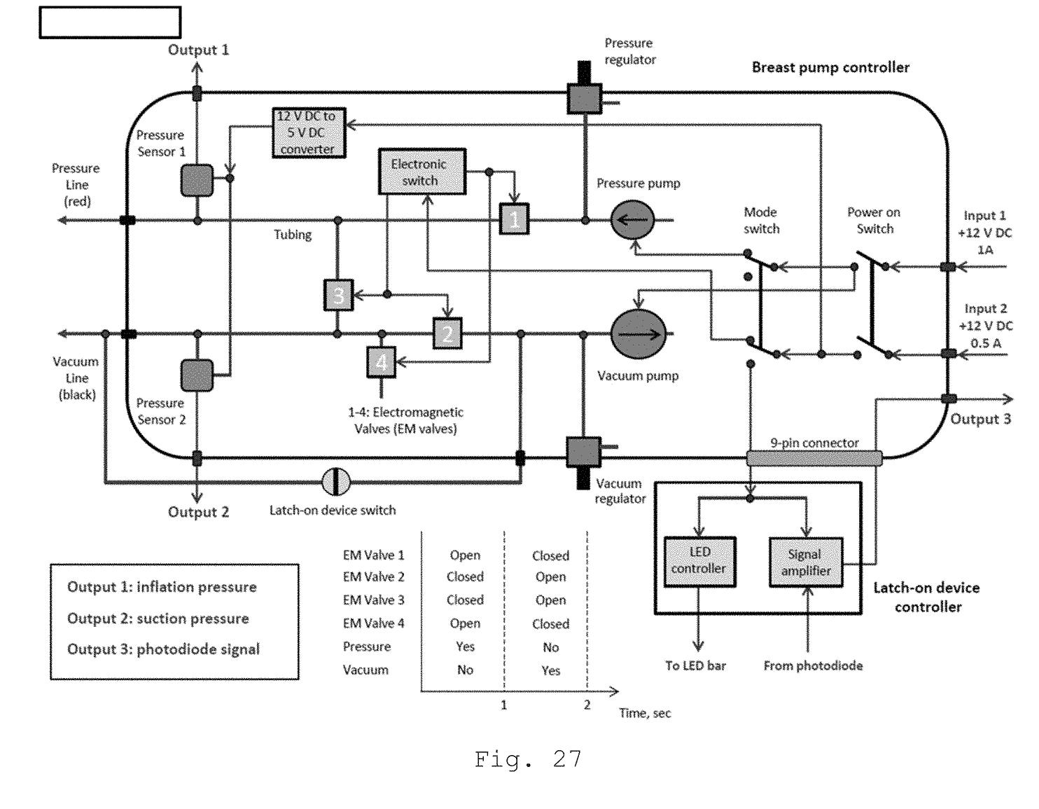

Referring to FIG. 24, the device 1300 in shown schematically during operation. Cup portion 1301 fits against the breast 1503 around the nipple. As the nipple is drawn into conical lumen 1302, it interrupts the light passing between photodiode 1303 and LED array 1304. A vacuum source 1502 is coupled to vacuum outlet 1306 and to pressure sensor 1503. Power supply 1504 provides power to pressure sensor 1503 and to amplifier 1505. Analog to digital converter 1506 converts the signals from the pressure sensor 1503 and the amplifier 1505 into digital signals suitable for recording by computer 1507. Exemplary output data is shown in FIG. 25, comprising coordinated pressure and length measurements.

Device 1300 is useful in experimental studies to explore the variability of the teat length during latch-on for a variety of subjects. This data in turn is useful to improve performance of breastfeeding pumps, for example, by calibrating the pressure of the breast pumps disclosed herein. Device 1300 is further useful for diagnosis of mothers with breastfeeding complications. It is useful for adjusting mother-specific breastfeeding pumps. It is useful for basic science and clinical applications, such as measuring nipple elasticity over the course of lactation, breast engorgement to monitor early lactation and help determine efficacy of early feeding and treatment, and objectively monitor inverted nipples.

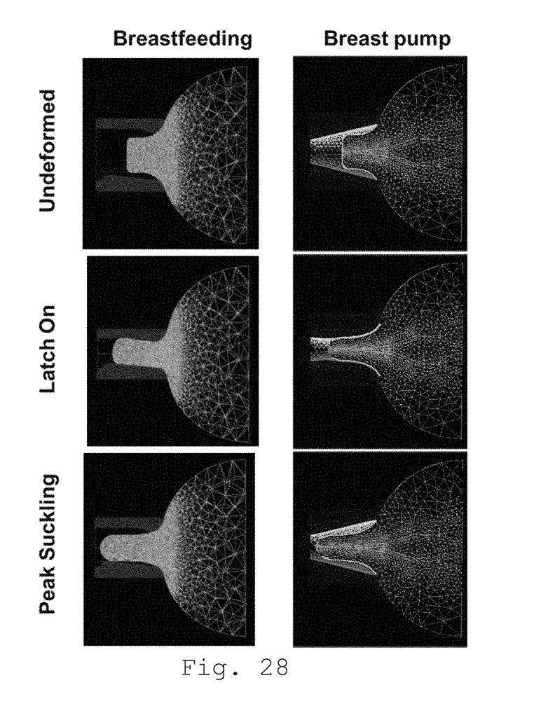

FIG. 26 depicts an exemplary latch-on meter controller. This controller works on 12V DC and contains a vacuum pump as well as a pressure sensor, LED, and photodiode as set forth in further detail above. FIG. 27 depicts an exemplary combination controller, suitable to control both the breast pumps described herein, as well as the diagnostic devices described herein. In some embodiments, the breast pump controller can be configured to provide multiple pressure and vacuum lines to support two or more breast pumps concurrently. In this manner, one user may use two breast pumps at the same time.

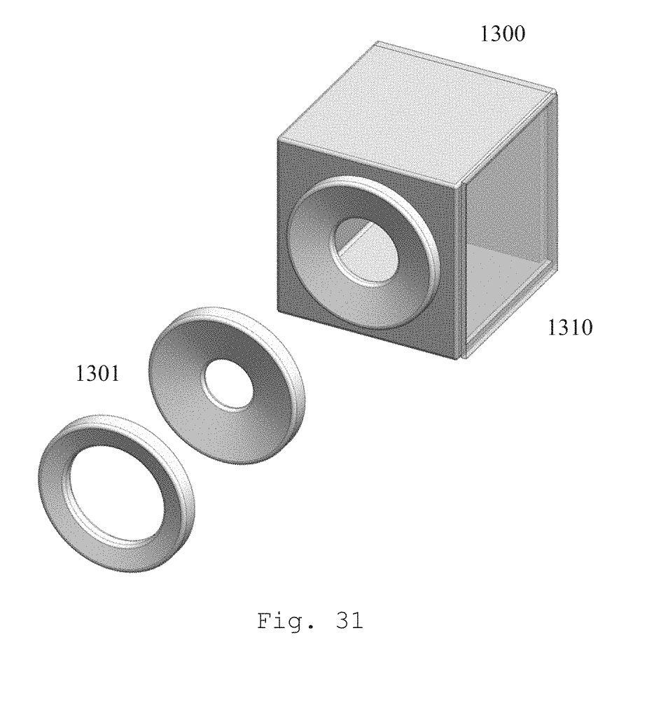

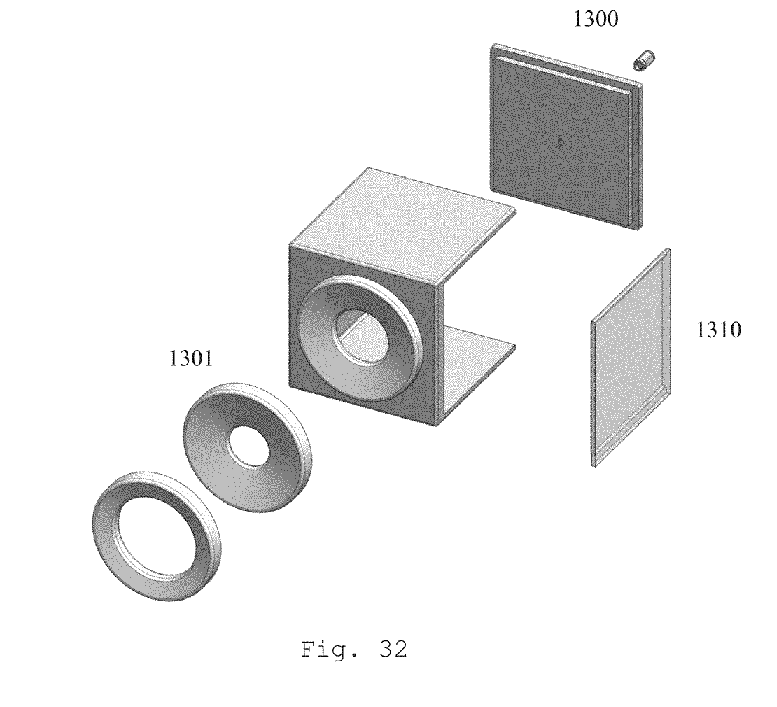

In another embodiment, as shown in FIGS. 31 and 32, a Latch-On meter is provided to measure the correct pressures required to latch on the breast. This pressure may vary between mothers of different ethnicities and body dimensions. In the device 1300′ of FIGS. 31 and 32, cup portion 1301′ fits against the breast around the nipple. As the nipple is drawn into device 1300 though cup portion 1301′, a vacuum source (not shown) applies a vacuum pressure on the breast in order to extend the nipple-areola, similar to latch-on experience. In some embodiments, the device has multiple surfaces defining an interior cavity, in which a vacuum source exists. The length of nipple extension is analyzed from images taken with a camera. In some embodiments the device is operatively engaged to the camera. In other cases, the camera a separate device and images are taken via the transparent wall 1310 of the device. via the transparent window 1310. The device will be connected with a tube to a control box equipped with a vacuum pump similar to the device 1300 with the photodetector.

Using the herein described diagnostic devices, the breast pumps of the present disclosure may be adjusted to the specific characteristics of an individual's breast. The diagnostic devices may be used to provide a quick evaluation of the sub-atmospheric pressures needed for optimal latch-on and efficient milk extraction from the breast. In some embodiments, the breast pump is adjusted by providing a customized insert with an appropriate size and shape. In some embodiments, the breast pump is adjusted by providing the optimal pressure and vacuum for the individual.

Moreover, an individualized bottle nipple may be created to fit the physical performance of an individual's nipple/breast. For example, the diagnostic devices described herein may be used for a quick evaluation of the sub-atmospheric pressures needed for extending the nipple/areola for optimal latch-on. Then, a lookup table based on computational simulation provides the best polymer for casting an individualized nipple that will function in a mechanically similar manner. This nipple allows the infant to feed on a breast or bottle in nearly identical fashion, reducing nipple confusion and other drawbacks of bottle feeding. Similarly, an individualized nipple shield may be created that enables the natural latch-on and nutritive feeding by infants with difficulty latching or maintaining attachment to the bare breast.

While the disclosed subject matter is described herein in terms of certain exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

What is claimed is:

1. A system for milk expression, comprising:
a first device comprising a conical lumen adapted to receive a nipple and an inflatable element arrayed on an interior surface of the conical lumen, the inflatable element operatively engaged to a pressure source adapted to successively inflate and deflate the inflatable element according to a first sinusoidal wave pattern; and
a second device comprising a receptacle operatively engaged to a vacuum source in fluid communication with a distal end of the conical lumen, wherein the vacuum source is adapted to provide fluctuations of negative pressure according to a second sinusoidal wave pattern, wherein the first and second sinusoidal wave patterns are concurrent and the same over time.

2. The system of claim 1, further comprising:
a controller, wherein the controller adjusts positive pressure output from the pressure source at a first predetermined frequency and negative pressure from the vacuum source at a second predetermined frequency.

3. The system of claim 2, wherein the first and second predetermined frequencies are concurrent.

4. The system of claim 2, wherein the controller is housed within the second device.

5. The system of claim 2, wherein the controller is remote from the first or second device.

6. The system of claim 1, wherein the pressure source is physically or operatively connected to the inflatable element by tubing.

7. The system of claim 1, wherein the vacuum source is physically or operatively connected to the conical lumen by tubing.

8. The device of claim 1, wherein the inflatable element cyclically inflates and deflates over a period of time.

9. The system of claim 1, wherein the vacuum source exerts negative pressures in the conical lumen of about −20 to −40 mmHg.

10. The system of claim 9, wherein the amount of negative pressure exerted in the conical lumen periodically increases and decreases over time.

11. The system of claim 9, wherein the amount of negative pressure exerted in the conical lumen is cyclical.

12. The system of claim 1, wherein the first device is wearable.

13. The system of claim 1, wherein the inflatable element comprises silicone sleeve.

15. The system of claim 1, wherein the receptacle is in fluid communication with the distal end of the conical lumen.

16. The system of claim 1, wherein the first device is configured to detachably couple to the second device.

17. The system of claim 16, wherein the first device includes a breast cup portion having a concave portion extending from an exterior circumference of a proximal end of the conical lumen and the second device is a bottle, wherein the conical lumen is configured to detachably couple to the bottle, and further wherein the pressure source is operatively connected to the breast portion by a first tubing and the vacuum source is operatively connected to the breast cup by a second tubing, wherein the second tubing is physically connected to the bottle.

18. A system for milk expression, comprising:
a first device comprising a conical lumen adapted to receive a nipple and an inflatable element arrayed on an interior surface of the conical lumen, the inflatable element operatively engaged to a positive pressure source adapted to successively inflate and deflate the inflatable element according to a first sinusoidal wave pattern; and
a second device in fluid communication with the conical lumen, wherein the second device comprises a receptacle, and
a vacuum source operatively engaged to the conical lumen, wherein the vacuum source is adapted to provide fluctuations of negative pressure according to a second sinusoidal wave pattern, wherein the first and second sinusoidal wave patterns are concurrent and the same over time.

19. The system of claim 18, wherein the pressure source and the vacuum source are operatively engaged to a controller adapted to regulate the positive and negative pressures from the pressure source and vacuum source, respectively.

20. The system of claim 19, wherein the controller is communicatively coupled to a computer system through a data acquisition module.
